(19) United States
(12) Patent Application Publication
(10) Pub. No.: US 2022/0016328 A1
(43) Pub. Date: Jan. 20, 2022

(54) COMBINATION WEARABLE AND STATIONARY DIALYSIS SYSTEM WITH DETACHABLE CANISTERS

(71) Applicant: Victor Gura, Beverly Hills, CA (US)

(72) Inventor: Victor Gura, Beverly Hills, CA (US)

(21) Appl. No.: 17/305,921

(22) Filed: Jul. 16, 2021

Related U.S. Application Data
(60) Provisional application No. 63/052,565, filed on Jul. 16, 2020.

(51) Int. Cl. A61M 1/36; B01D 15/08

(52) U.S. Cl. CPC A61M 1/3679; A61M 1/3417; B01D 15/08; A61M 1/367

(57) ABSTRACT

Various embodiments disclosed relate to a system and methods for hemodialysis, including detachable sorbent cartridge canisters. The present disclosure can include a system for hemodialysis having a first dialysis module including a dialyzer, a blood circuit configured to receive blood from a patient, circulate the blood through the dialyzer, and return cleaned blood to the patient, a dialysate circuit configured to circulate dialysate through the dialyzer and remove impurities from the blood, and a first canister detachably coupled to dialysate circuit, the canister comprising a first sorbent.

### PRIORITY APPLICATION

This application claims priority to U.S. Provisional Application Ser. No. 63/052,565, filed Jul. 16, 2020, the disclosure of which is incorporated herein in its entirety by reference.

### BACKGROUND

Hemodialysis can be a renal replacement therapy used by patients who have end stage renal disease (ESRD). These patients can no longer rely upon their kidneys to provide desired removal of waste from the blood. Hemodialysis can involve extracorporeal removal of toxins from a patient's blood using a dialyzer, where the toxins diffuse across a semipermeable membrane in the dialyzer to a dialysate solution due to a concentration gradient across the membrane.

### SUMMARY OF THE DISCLOSURE

Additional aspects and advantages of the present disclosure will become readily apparent to those skilled in this art from the following detailed description, wherein only some examples of the present disclosure are shown and described, simply by way of illustration of the several modes or best mode contemplated for carrying out the present disclosure. As will be realized, the present disclosure is capable of other and different examples, and its several details are capable of modifications in various obvious respects, all without departing from the disclosure. Accordingly, the drawings and description are to be regarded as illustrative in nature, and not as restrictive.

In an example, a system for hemodialysis can include a first dialysis module. The first dialysis module can include a dialyzer, a blood circuit configured to receive blood from a patient, circulate the blood through the dialyzer, and return cleaned blood to the patient, a dialysate circuit configured to circulate dialysate through the dialyzer and remove impurities from the blood, and a first canister detachably coupled to dialysate circuit, the canister comprising a first sorbent for removing toxins from the blood.

In an example, a method of hemodialysis using a hemodialysis system, can include continuously removing a first set of toxins from blood over a combined cycle with a first dialysis module, selectively removing a second set of toxins from the blood over a portion of the combined cycle with a second dialysis module, wherein removing the first set and the second set of toxins comprises using one or more detachable canisters fluidly connectable to the first dialysis module, the second dialysis module, or both.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic diagram of a portable hemodialysis system in an example.

FIG. 2 is a schematic of a stationary hemodialysis module in an example.

FIGS. 3A-3B are schematic diagrams of a detachable sorbent cartridge canister for use with a hemodialysis system.

FIGS. 3C-3H are perspective views of a detachable sorbent cartridge canister for use with a hemodialysis system.

FIGS. 4A-4D are perspective exploded views of detachable sorbent cartridge canisters for use with a hemodialysis system.

FIGS. 5A-5E are views of connectors for detachable sorbent cartridges.

FIG. 6 is an exploded view of a series of detachable canisters.

### DETAILED DESCRIPTION

While some examples of the invention have been shown and described herein, it will be obvious to those skilled in the art that such illustrations are provided by way of example only. Numerous variations, changes, and substitutions will now occur to those skilled in the art without departing from the invention. It should be understood that various alternatives to the examples of the invention described herein may be employed in practicing the invention.

Disclosed is a system and methods of hemodialysis. The system includes a portable module that can be comfortably worn by the patient, such as during normal daytime activity. The portable module can plug into a stationary module having additional sorbents, such as for removal of urea. The portable and stationary modules can share a pump, dialysate circuit, and blood circuit. The system can optionally include an auxiliary pump and ultrafiltrate module, which is some cases can be detachable to allow for improved portability of the portable module.

Hemodialysis, commonly called kidney dialysis or simply dialysis, is a process of purifying the blood of a person whose kidneys are not working normally. This type of dialysis achieves the extracorporeal removal of waste products such as creatinine and urea and free water from the blood when the kidneys are in a state of kidney failure. Hemodialysis can be an outpatient or inpatient therapy. Routine hemodialysis is conducted in a dialysis outpatient facility, either in a purpose built room in a hospital or a dedicated, stand-alone clinic, or at home. Less frequently hemodialysis is done at home. Dialysis treatments in a clinic are initiated and managed by specialized staff made up of nurses and technicians; dialysis treatments at home can be self-initiated and managed or done jointly with the assistance of a trained helper.

Conventional hemodialysis has a number of disadvantages, such as restricted independence, as people undergoing this procedure cannot travel around due to suppliers availability and being tethered to a large stationary device during treatment; requires high water quality; a large quantity of water; and continuous source of electricity, typically provided by a power plug connected to an outlet; requires reliable technology like dialysis machines; requires healthcare provider such as nurses and technicians having more knowledge of the complicated procedure and equipment; requires ongoing and repetitive time to set up and clean dialysis machines.

Additionally, hemodialysis often involves fluid removal through ultrafiltration, as most patients with renal failure pass little or no urine. Side effects caused by removing too much fluid or removing fluid too rapidly can include low blood pressure, fatigue, chest pains, leg-cramps, nausea and headaches. These symptoms can occur during the treatment and can persist post treatment; they are sometimes collectively referred to as “dialysis hangover” or “dialysis washout.” The severity of these symptoms is usually proportionate to the amount and speed of fluid removal. However, the impact of a given amount or rate of fluid removal can vary greatly from person to person and day to day.

Conventional hemodialysis is usually done three times per week, for about 3-4 hours for each treatment, during which the patient's blood is drawn out through a tube at a rate of 200-400 mL/min. The tube is connected to a 15, 16, or 17 gauge needle inserted in the dialysis fistula or graft, or connected to one port of a dialysis catheter. The blood is then pumped through the dialyzer, and then the processed blood is pumped back into the patient's bloodstream. Optionally in any example, the blood can be pumped to the patient through another tube connected to a second needle or port. Optionally in any example, the blood can be pumped to the patient through a dual lumen catheter at the same location where blood is removed from the body, such as to allow one access point to the body to both remove and return blood in two separate lumens.

During the procedure, the patient's blood pressure is closely monitored, and if it becomes low, or the patient develops any other signs of low blood volume such as nausea, the dialysis attendant can administer extra fluid through the machine. During the treatment, the patient's entire blood volume (about 5000 cc) circulates through the machine every 15 minutes. During this process, the dialysis patient is exposed to a week's worth of water for the average person. Daily hemodialysis is typically used by those patients who do their own dialysis at home. It is less stressful (gentler) but does require more frequent access. Daily hemodialysis is commonly done for 2 hours six days a week. The procedure of nocturnal hemodialysis is similar to conventional hemodialysis except it is typically performed three to six nights a week and between six and ten hours per session while the patient sleeps.

Described herein are systems and methods related to a dialysis system which can include both a first dialysis module which performs dialysis functions during a period when the patient desires to be mobile, such as during the daytime, and a second dialysis module configured to perform dialysis functions during a period of time when mobility is not as important to the patient, such as during the nighttime. The system can also include a detachable auxiliary module for collection of ultrafiltrate and liquid removal.

The patient can alternate use of the first dialysis modules alone, and the second dialysis module in conjunction with the first dialysis module, such as during a 24-hour period. The patient can selectively attach or detach the auxiliary module to allow for removal of fluid at various parts of the day or attach the auxiliary module for an extended period of time when stationary.

The first dialysis module can be worn by the patients, such as under their clothes. The first dialysis module is lightweight and/or compact to facilitate transport of the system such that patients can maintain activities of daily life. The removal or addition of the auxiliary module with an ultrafiltrate collector can allow even more patient movement and mobility during the day.

Meanwhile, the second dialysis module connects into the first dialysis module to create a stationary dialysis system, configurable to provide additional toxin removal functions. The stationary dialysis system is stationary, for example being configured to be positioned on a support, such as during nighttime when the patient is stationary. This can allow a patient to be mobile during the day while maintaining overall desired removal of toxins but would constrain movement at night.

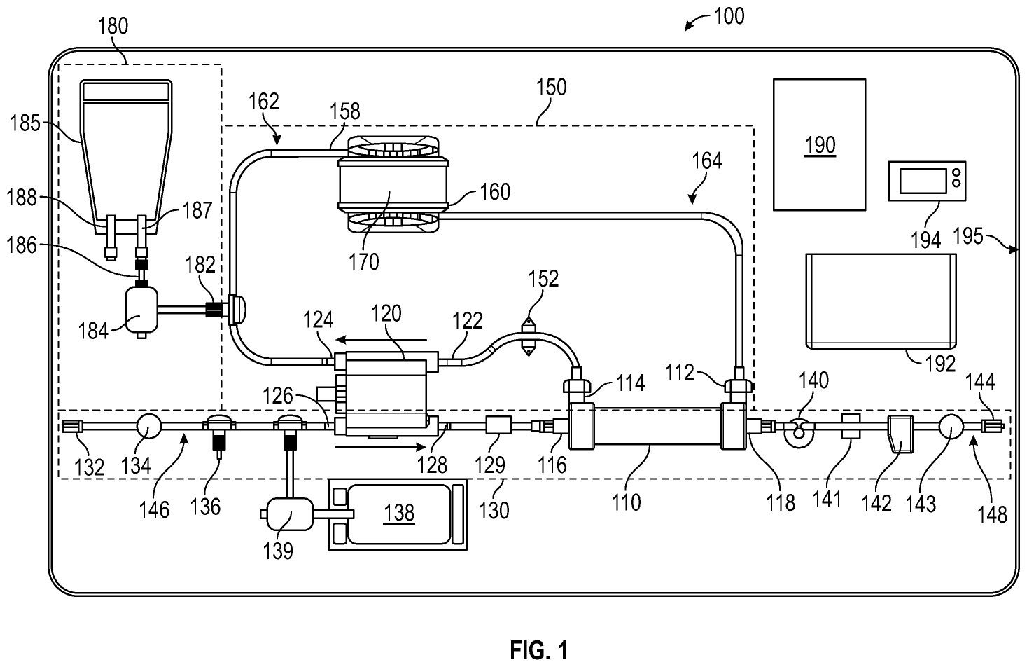

FIG. 1 is a schematic diagram of a portable first dialysis module 100 with a detachable auxiliary pump module 180. The portable hemodialysis system (“first dialysis module”) 100 can be configurable to be transported by a patient, for example to perform toxin removal functions while a patient is mobile, such as during the daytime. The first dialysis module 100 can be lightweight and compact in size to provide desired patient mobility. For example, the first dialysis system can be of a weight less than 5 lbs., less than 4 lbs., less than 3 lbs., less than 2.5 lbs., less than 2 lbs., less than 1.5 lbs., or less than 1 lb. The first dialysis module 100 can be configurable to be worn by the patient while the patient is going about his or her daily business without requiring tethering to external power sources or external components. For example, the first dialysis module 100 can be a wearable artificial kidney. The first dialysis module 100 can be coupled to a belt such that the first dialysis module 100 is worn by the patient. The first dialysis module 100 can be worn by the patient, but otherwise untethered.

The first dialysis module 100 can include a dialyzer 110, a pump 120, a blood circuit 130, a dialysate circuit 150, a canister 170, auxiliary module 180, control unit 190, power source 192, user interface 194, and power jack 195.

The dialyzer 110 can have a dialysate input 112, a dialysate output 114, a blood inlet 116, and a blood outlet 118. Dialysate can flow through the dialyzer 110 in a first direction while the blood flows through the dialyzer 110 in a counter current flow. Counter current flow can maximize the gradient between the dialysate circuit 150 and the blood circuit 130, therefore maximizing exchange across the dialyzer 110 membrane. Toxins from the blood flow can diffuse into the dialysate across semi-porous membranes of the dialyzer 110 as the blood and dialysate flow across opposing surfaces of the semi-porous membranes. In an example, blood flow can travel in a clockwise fashion through the blood circuit 130, while the dialysate can flow in a counterclockwise fashion through the dialysate circuit 150.

In dialysis systems, some toxins be removed at a steady rate over a 24-hour period, but generally not faster. However, some toxins can be removed over shorter periods of time without negative consequences to the patient. There are two types of toxins: those bound to protein; and free toxins. Free toxins are generally considered to be more toxic. Examples of toxins that require removal over 24 hours include p-cresyl and indoxyl sulfate. These are part of a group of toxins called protein bound toxins (P-BUTS). The free form, which is the only toxic one, comes out in the urine, keeping its level low in a healthy patient. In dialysis the free fraction comes out on dialysis and the level of the free toxin is also low, however, as soon as the patient is on a dialysis machine, the protein bound toxins re-equilibrate with the free fraction, that comes up again to toxic levels. There are about 25 known P-BUTS. The first dialysis module 100 can be configured to remove toxins at a steady rate over a 24-hour period.

The pump 120 can have a dialysate input 122, a dialysate output 124, a blood inlet 126, and a blood outlet 128. The pump 120 can be a side-to-side pulsatile pump. The side-to-side pulsatile pump 120 can be powered by a battery, including a rechargeable battery, and/or by an electrical wall outlet. For example, the side-to-side pulsatile pump 120 can be powered by a battery to enable transport of the pump 120, thereby facilitating transport of the dialysis system which incorporates the pump 120, such as first dialysis module 100. An example of such a side-to-side pump is disclosed in U.S. patent application Ser. No. 15/890,718; the entire contents of which are incorporated herein by reference.

The side-to-side pulsatile pump 120 can be configured to retain a blood tubing permitting the flow of blood therethrough from the patient, and a dialysate tubing permitting flow therethrough of dialysate, within a pump casing. The pump can include a compression disc configured to provide side-to-side motion to apply a first pressure to the blood ventricle tubing and a second pressure to the dialysate ventricle tubing in alternate fashion. This can allow for alternating pumping of the blood circuit 130 and the dialysate circuit 150. In some cases, the pump can be driven by a motor and gear box. The pump 120 can create a pulsatile flow where the blood pulses are out of phase with the dialysate pulses, such that, for example, the peak of the blood pulse is 90 to 180 degrees out of phase with the peak of the dialysate flow.

One or more side-to-side pulsatile pumps described herein can be configured to provide desired pumping volume for both blood and dialysate, while reducing or eliminating problems associated with known pumps. Optionally in any example, one or more side-to-side pulsatile pumps described herein can provide pumping volumes of greater than about 35 milliliter per minute (mL/min). Optionally in any example, a dialysis system using a side-to-side pulsatile pump can provide a flow rate of dialysate of about 100 mL/min.

The blood circuit 130 can have an inlet 132, a flow control element 134, a saline flush 136, a blood thinner hookup 138 with pump 139, a bubble filter 140, a bubble detector 141, a flow sensor 142, a flow control element 143, and an outlet 144. The blood circuit can contain first portion 146 and second portion 148. The first portion contains undialyzed blood, the second portion contains dialyzed blood. The blood circuit 130 can, for example, be made of tubing or other conduit suitable for flow of blood. The flow control elements 134, 143, can be elements such as valves, clamps, or other elements that allow for turning off and on blood flow, or otherwise controlling flow rates through the blood circuit 130.

In the first portion 146 of the blood circuit 130, the inlet 132 can be configured for attachment to a patient. In some cases, the inlet 132 can be a blood thinner infusion inlet, such as for adding blood thinner to the blood flow to prevent blood clots from forming within the blood circuit 130 of the first dialysis module 100. In some cases, such as shown in first dialysis module 100, the blood thinner hookup 138 can be separate from the inlet. Such a hookup can be connected to a blood thinner reservoir (not shown). Example blood thinners can include heparin, or more specifically, low molecular weight heparins, direct thrombin inhibitors, danaparoid, ancrod, r-hirudin, abciximab, tirofiban and argatroban, among others known to those skilled in the art. Optionally in any example, a blood thinner infusion inlet can be positioned elsewhere on the blood circuit 130, such as after the pump 120. The infusion of one or more blood thinners into the blood circuit 130 can be actuated, for example, by the pump 139.

The first portion 146 can include flow of blood from the patient that has not yet been treated for toxins. The second portion 148 can include flow of blood back to the patient that has been treated for toxins. In the first portion 146, the blood circuit 130 can allow for flow of blood from the inlet 132 through the pump 120 to the dialyzer 110 via blood inlet 116, where toxins can be removed. In the second portion 148, upon exiting the dialyzer at blood outlet 118, blood can flow towards the outlet 144 towards the patient. The blood flow can run through a number of optional components which may be included in any example, such as the bubble filter 140 and bubble detector 141, the flow sensor 142, or other sensors or filters.

The bubble filter 140 and bubble detector 141 can be in fluid communication with the blood flow exiting the dialyzer 110 such that presence of air bubbles within the blood is detected and communicated to the control unit 190. The control unit 190 is configurable to pause and/or power off the first dialysis module 100 upon detection of air bubbles within the blood flow.

The flow sensor 142 can be in line or parallel to the blood circuit 130, such as in second portion 148 of the blood circuit. The flow sensor 142 can be configured to measure the rate at which blood is flowing through the first dialysis module 100. Optionally in any example, one or more flow sensors can be alternatively or additionally be on the dialysate circuit for measuring flow of dialysate. The flow sensor 142 can be a mechanical flow meter, a pressure-based flow meter, a variable area flow meter, an optical flow meter, combinations thereof, or other type of flow sensors.

The flow sensor 142 on the blood circuit 130 can detect the volume of blood moving through the blood circuit over a given time period. This information can be communicated to the control unit 190, which is turn can monitor the flow of blood through the circuit. If the blood flow is outside of a normal range, the control unit 190 can alter the movement of the dialyzer 110 and pump 120 to change the flow of blood and/or dialysate through the module 100. For example, if the blood flow is too slow, it may indicate a clot or blockage, which may need to be addressed. Optionally in any example, a change in flow may trigger an alarm such as an audible, visual, tactile, or other indicia to the user. If the blood flow is too quick, the control unit 190 can slow the mechanism of the pump 120 to modulate the flow of fluid in the module 100 accordingly.

Optionally in any example, the first dialysis module 100 can additionally include a pH sensor 129 tied to the control unit 190 such as to test for ammonia in the blood circuit. The pH sensor 129 can be, for example, positioned between the dialyzer 110 and the outlet 144. Since ammonia is such a strong base, the pH sensor 129 can provide a safety mechanism to detect when the canisters are no longer effective for removing ammonia from the system. The optional pH 129 sensor can be in fluid communication with the blood flow exiting the dialyzer 110 such that the presence of ammonia within the fluid is detected and communicated to the control unit 190. The control unit 190 can be configurable to pause and/or power off the first dialysis module 100 upon detection of ammonia within the blood flow, or otherwise trigger an alarm. The pH sensors 129 can be, for example, a combination pH sensor, a differential sensor, a laboratory sensor, a process pH sensor, or other type of pH sensor.

The dialysate circuit 150 can include a blood detector 152 and connections 158, 160 to the canister 170. The dialysate circuit can include first portion 162 and second portion 164. The dialysate circuit 150 is a sterile dialysate circuit for flow of dialysate therethrough. The dialysate circuit 150 can allow flow of a dialysate through the dialyzer 110 and the pump 120, through the canister 170, and back to the dialyzer 110. The dialysate circuit 150 can, for example, be made of tubing or other conduit suitable for flow of dialysate.

The first portion 162 of the dialysate circuit 150 can include a blood detection access port connecting the dialysate circuit 150 to the blood detector 152. The blood detection access port can be coupled the blood detector 152, such that presence of blood in the dialysate exiting the dialyzer 110 can be detected. In some cases, breakage in the membranes of the dialyzer 110 can result in blood entering the dialysate flow. The blood detector 152 can be in communication with the control unit 190 such that the control unit 190 will pause and/or power off the first dialysis module 100 upon detection of blood in the dialysate, or otherwise cause an alarm to be initiated to the user.

Dialysate can be driven by the pump 120 from the dialyzer 110 through dialysate output 114 into the first portion 162 of the dialysate circuit towards the canister 170 via connection 158. In some cases, the first portion 162 of the dialysate circuit can be connected to the auxiliary module 180. The dialysate can be driven through the canister 170, where the sorbent in the canister 170 treats the dialysate, and then the dialysate flows out the connection 160 to the second portion 164 of the dialysate circuit. In the second portion 164 of the dialysate circuit, the dialysate can be driven from the canister 170 back towards the dialyzer, where the dialysate can enter the dialyzer 110 through the dialysate input 112.

In first dialysis module 100, which is designed to be mobile, one lighter weight canister 170 can be used. The canister 170 include a sorbent that can be, for example, charcoal. Optionally in any example, the canister 170 can include a sorbent configured to remove one or more of organic uremic metabolites and heavy metals. Optionally in any example, the canister 170 sorbent is configured to remove one or more of creatinine, uric acid and P2 micro globulins, p-cresol, indoleacetic acid and hippurate. In an example, the canister 170 sorbent comprises activated carbon, such as charcoal. The dialysate exiting canister 170 is regenerated dialysate, such that dialysate entering the dialyzer 110 is cleaned dialysate.

The auxiliary module 180 can be a detachable module for removal of ultrafiltrate. In some cases, the first portion 162 of the dialysate circuit can be connected to the auxiliary module 180 through the ultrafiltrate outlet port 182. The ultrafiltrate outlet port 182 can be, for example, a spike for fluid-tight connection and detachment of the auxiliary module 180 as desired. The auxiliary module 180 can include ultrafiltrate outlet port 182, a pump 184, and an ultrafiltrate collector 185.

The auxiliary module 180 can be removeable or attachable to the first dialysis module 100, using a connector element. The connector element can be on the auxiliary module 180, and releasably connect with a cooperating connector element on the first dialysis system. For example, a surgical spike could also be on the auxiliary module 180, and the first dialysis module 100 can have a receptacle such as a resilient rubber seal for receiving the spike. In some cases, the connector, such as a surgical spike, can be located on the first dialysis module 100 and the cooperating connector is located on the auxiliary module.

The ultrafiltrate from the dialysate can exit the dialysate circuit 150 through the ultrafiltrate outlet port 182 and can be collected within the ultrafiltrate collector 185 which can be a bag, canister or any other reservoir for collecting the ultrafiltrate. The ultrafiltrate collector 185 can include an ultrafiltrate inlet port 186 configured to be coupled a first fluid channel 187. The first fluid channel 187 can be configured to provide fluid communication between the ultrafiltrate pump 184 and the ultrafiltrate collector 185. A second fluid channel 188 can be coupled to the ultrafiltrate pump 184 to provide fluid communication between the ultrafiltrate pump 184 and the dialysate circuit 150. The ultrafiltrate pump 184 can be used to control flow of ultrafiltrate from the dialysate circuit 150 into the ultrafiltrate collector 185. The ultrafiltrate pump 184 can be a micro-pump. Removal of ultrafiltrate can provide removal of water and sodium from the dialysate. For example, the ultrafiltrate removal rate can be maintained at a physiological rate in order to reduce or avoid blunt hemodynamic changes.

Optionally in any example, the dialysate circuit 150 of the first dialysis module 100 can include one or more points at which optional electrolyte is infusible into the dialysate flow. One or more types of optional electrolyte solutions can be added into the dialysate flow to facilitate maintaining electrolyte homeostasis. For example, one or more of optional electrolyte supplement solutions, such as electrolyte supplement solutions comprising sodium bicarbonate, calcium, and/or magnesium, can be infused into the dialysate flow at one or more optional electrolyte infusion points.

Optionally in any example, the second portion 164 of the dialysate circuit can include one or more electrolyte infusion ports with electrolyte reservoirs (not shown). The electrolyte reservoir can retain an electrolyte solution. Optionally in any example, the electrolyte solution can be used to adjust the pH of the dialysate. The electrolyte solution can be, for examples, sodium bicarbonate solution. The electrolyte solution can be infused into the dialysate flow via an electrolyte infusion port. Flow of the electrolyte solution into the dialysate flow can controlled by an electrolyte solution pump. Such an electrolyte solution pump can be configured to pump up to about 5 milliliters per hour (mL/hr.), or for example from about 1 mL/hr. to about 2 mL/hr., up to about 5 mL/hr.

The control unit 190 can be in electrical communication with one or more components of the first dialysis module 100. For example, the control unit 190 can be in communication with the bubble detector 141 and the blood detector 152 such that an alarm is initiated when air bubbles are detected in the blood flow and/or blood is detected in the dialysate flow. Optionally in any example, the control unit 190 is configured to pause and/or power down the first dialysis module 100 upon detection of air bubbles in the blood flow and/or blood in the dialysate flow. Optionally in any example, the control unit 190 is configured to control the pump 120 to provide desired flow of blood and/or dialysate through the first dialysis module 100. The control unit 190 can control one or more optional pumps configured to control flow of electrolyte into the dialysate, blood thinner into the blood flow, and/or ultrafiltrate from the dialysate.

The power source 192 can be a portable power source, such as a battery or a rechargeable battery, connected to the first dialysis module 100. In some cases, the power source 192 can additionally include an option to plug into a wall outlet.

The user interface 194 can allow for the patient to see status updates or monitor functioning of the dialysis first dialysis module 100. The user interface 194 can include, for example, buttons, a screen, lights, or other indicia that can convey whether the system is functioning properly.

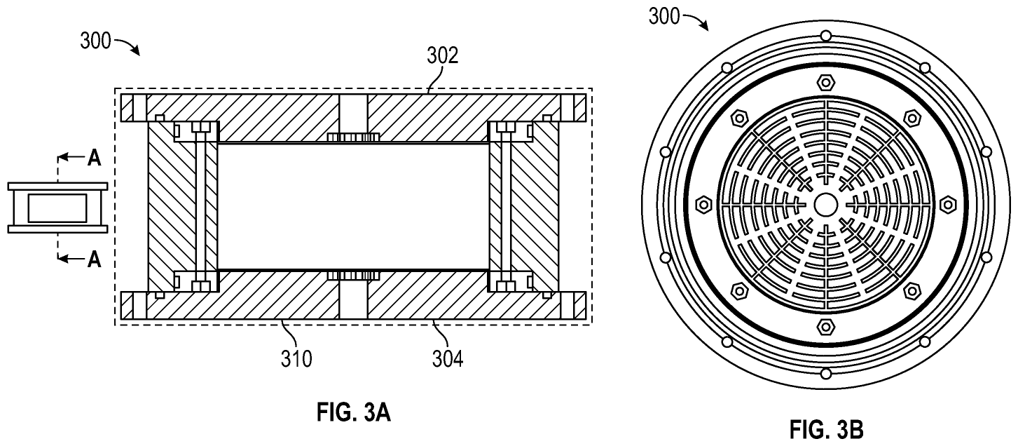

The first dialysis module 100 is lightweight and wearable by a patient during the daytime, or when he or she is going about normal daily activities. The first dialysis module 100 can be worn, for example, as a belt, shown and discussed with reference to FIGS. 3A-3B, 4A-4C, and 5A-5C below. The light-weight first dialysis module 100 can include an activated carbon cartridge configured to adsorb various toxins from the dialysate. The activated carbon is configured to remove one or more of creatinine and β2 micro globulins, p-cresol, indoleacetic acid, hippurate, and heavy metals, from the dialysate. Optionally in any example, the first dialysis module is not configured to remove urea from the blood flow. In the first dialysis module alone, worn in a mobile configuration, a charcoal sorbent, or other sorbents known in the art, can continuously removes the free fraction of the P-BUTS over 24 hours.

The detachable auxiliary module 180 can allow for a light weight and comfortable system with flexibility for regular, but not necessarily continuous, removal of sodium and water. The patient can strategically and selectively plug into the auxiliary module 180 as needed to expel fluid. This can be monitored and timed according to the patient's needs to avoid hemodynamic problems.

The first dialysis module 100 and the auxiliary module 180 can be combined with the second dialysis module 200 to allow for removal of additional toxins over a shortened, stationary, period of time.

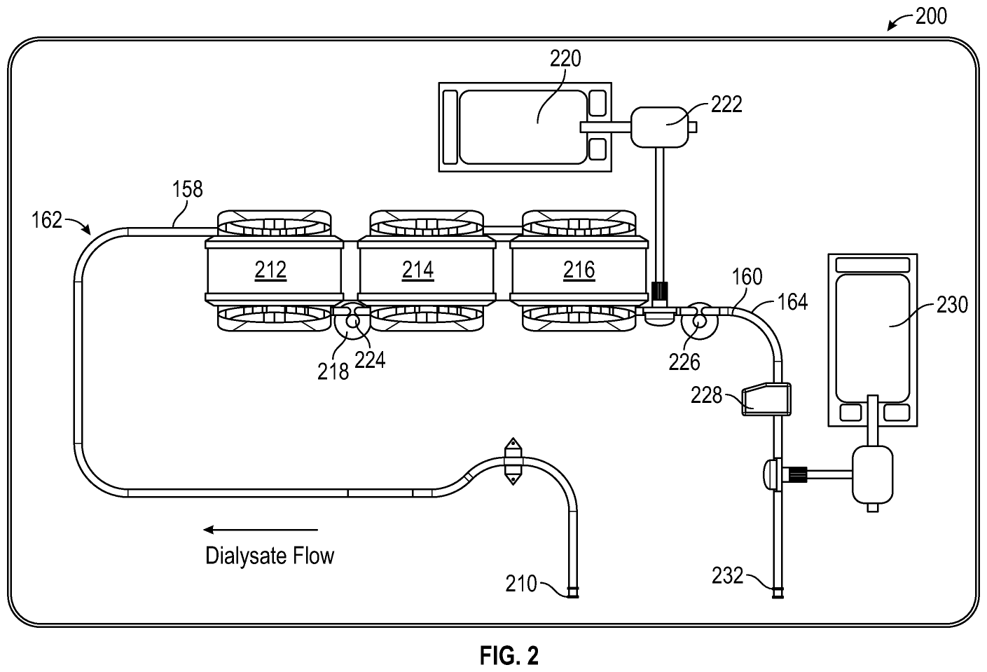

FIG. 2 is a schematic of a stationary hemodialysis module 200 for use with a first stationary dialysis module 100, shown in FIG. 1. The stationary hemodialysis module (“second dialysis module”) 200 can include a dialysate inlet 210, sorbents 212, 214, 216, ammonia sensor 218, first electrolyte module 220 with pump 222, bubble filters 224, 226, flow sensor 228, second electrolyte module 230, and dialysate outlet 232.

The second dialysis module 200 is a module that plugs into the first dialysis module 100 of FIG. 1. The second dialysis module 200 does not perform hemodialysis alone. Instead, the dialysate inlet 210 and the dialysate outlet 232 are connected to the connections 158, 160 of the dialysate circuit 150 of the first dialysis module 100. This replaces or supplements the canister 170 from the portable first dialysis module 100 with the canisters 212, 214, and 216 from the second dialysis module 200. Optionally in any examples, the canister 170 from system 100 can be used with the second dialysis module 200, in addition to or instead of one of canisters 212, 214, 216.

When the second dialysis module 200 is plugged into the first dialysis module 100 for use by the patient, the dialysate can travel from the dialyzer 110 into the first portion 162 of the dialysate circuit 150, through the blood detector 152, through the pump 120, up past the auxiliary module 180, and into the dialysate inlet 210 of the second dialysis module 200. Optionally in any examples, the first dialysis module can recharge while plugged into the second dialysis module.

The dialysate can then be driven, by the pump 120, through the second dialysis module 200, from the dialysate inlet 210 through canisters 212, 214, 216 in sequence. The dialysate can travel through one or more of bubble filters 224, 226, ammonia sensor 218, and flow sensor 228, in addition to auxiliary electrolyte modules 220, 230, on its way to the dialysate outlet 232. Once the dialysate reaches the dialysate outlet 232 of the second dialysis module 200, it can be driven back into the dialyzer 110.

The second dialysis module can include a plurality of types of sorbent materials configured to regenerate the dialysate. For example, the second dialysis can include a plurality of canisters, each with its own sorbent material, to provide a plurality of types of sorbent materials. A patient can alternate use of the first dialysis module and a second dialysis module, for example for about 12 hours each during a 24 hour period, such that the patient remains mobile during the day while maintaining overall desired removal of toxins.

A sorbent material in the one or more canisters 212, 214, 216 can include sorbents such as carbon, charcoal, zirconium phosphate; hydrous zirconium oxide; metals or alloys containing zirconium; an organic and/or inorganic compound comprising zirconium; minerals comprising zirconium; or urease.

The canisters 212, 214, 216, can include a urea converter cartridge 212 and two other canisters 214, 216. The urea converter cartridge 212 can be configured to convert urea to ammonium carbonate, which in the presence of hydrogen ions generates carbon dioxide. For example, the urea converter cartridge can include urease. Optionally in any example, the urea converter cartridge 212 comprises one or more sorbent materials configured to adsorb toxins in the dialysate. The one or more sorbent materials can be configurable to adsorb ammonium, such as the ammonium generated by the degradation of urea into ammonium carbonate. Optionally in any example, the one or more sorbent materials are configurable to adsorb other cations, including cations of calcium, magnesium, and/or potassium. Optionally in any example, the urea converter cartridge 212 includes zirconium phosphate. For example, zirconium phosphate in the urea converter cartridge 212 can remove ammonium from the dialysate, along with calcium, magnesium and potassium cations, while releasing sodium and hydrogen ions.

Optionally in any example, the urea converter cartridge 212 comprises more than one distinct cartridge and/or distinct portions of cartridges. Optionally in any example, the urea converter cartridge 212 is configurable to be split into more than one distinct cartridge. The urea converter cartridge 212 can include one or more cartridges to retain the urea converter component, such as the urease, and one or more cartridges to retain the one or more sorbent materials. For example, the urea converter cartridge 212 can include a first cartridge configured to retain the urease and a second cartridge configured to retain the one or more sorbent materials. Optionally in any example, the urea converter cartridge 212 comprises more than one distinct portions, with one or more respective portions comprising the urease and one or more sorbent materials.

The first canister 214 can include a sorbent configurable to remove one or more heavy metals and/or one or more anions from the dialysate. For example, the first canister 214 sorbent can remove one or more of iron, mercury and aluminum. In some cases, the canister 214 is configured to remove one or more phosphate and sulfide anions. In some cases, the first canister 214 sorbent comprises hydrous zirconium oxide. For example, dialysate can flow through the urea converter cartridge 212 and into the first canister 214 where heavy metals, such as iron, mercury and aluminum, and phosphate and sulfide anions are removed from the dialysate, in exchange for acetate. Zirconium hydroxide binds phosphate and releases acetate, bicarbonate and sodium in small amounts. Zirconium phosphate removes ammonium, calcium, magnesium and potassium.

The second canister 216 sorbent can be configurable to remove one or more of organic uremic metabolites and heavy metals. In some cases, the second canister 216 sorbent is configured to remove one or more of creatinine, uric acid and β2 micro globulins, p-cresol, indoleacetic acid and hippurate. The second canister 216 can include activated carbon, such as charcoal. In some cases, the second canister 216 has characteristics similar to or the same as the canister 170 described with reference to FIG. 1.

Once the dialysate flows through the sorbent containing cartridges 212, 214, 216, the dialysate can be driven back through the second dialysis module 200 past the ammonia sensor 218, the first electrolyte module 220, the bubble filters 224, 226, the flow sensor 228, the second auxiliary module 230, and to the dialysate outlet 232, where the cleaned dialysate can return to the dialyzer 110. The dialysate exiting the canisters 212, 214, 216 is regenerated dialysate, such that dialysate flowing into the dialyzer 110 is cleaned dialysate which can be used to remove toxins from the blood of the patient. Since the dialysate system is sterile, ideally, the dialysate would be changed between each conversion from the first dialysis module 100, to the second dialysis module 200, or between each conversion from the second dialysis module 200, to the first dialysis module 100.

The second dialysis module 200 can have one or more ammonia sensors 218 in line with the dialysate circuit. The ammonia sensor 218 can be, for example, a pH sensor similar to the pH sensor of first dialysis module 100. The ammonia sensor can be tied to the control unit 190 to test for ammonia in the blood circuit. Since ammonia is such a strong base, the optional pH sensor provides a safety mechanism to detect when the canisters are no longer effective for removing ammonia from the system. The optional pH sensor is in fluid communication with the blood flow exiting the dialyzer 110 such that the presence of ammonia within the fluid is detected and communicated to the control unit 190. The control unit 190 can be configurable to pause and/or power off the first dialysis module 100 upon detection of ammonia within the blood flow.

Optionally, the second dialysis module 200 can have one or more bubble filters 224, 226 in line with the dialysate circuit. The bubble filters 224, 226 can be in fluid communication with the dialysate such that presence of air bubbles within the dialysate is detected and communicated to the control unit 190 control unit 190. The control unit 190 is configurable to pause and/or power off the first dialysis module 100 upon detection of air bubbles within the dialysate.

The auxiliary electrolyte modules 220, 230 can be in line with the dialysate circuit of the second dialysis module 200. The auxiliary electrolyte modules 220, 230, can provide saline or solution into the dialysate flow. The auxiliary electrolyte modules 220, 230 can include sodium bicarbonate, and is optional, when the first dialysis module 100 is primed with a primer solution containing bicarbonate (HCO3-). For example, when the dialysate circuit is initially primed with a primer solution, typically containing saline (or half-normal saline) and bicarbonate (HCO3-), the need for a separate electrolyte reservoir can be obviated.

The bulkier and heavier second dialysis module 200 can be configured to remove urea. For example, the urea converter of the second dialysis module 200 can decompose urea removed from the blood stream into ammonia and carbon dioxide. The second dialysis module can remove the ammonia and vent the carbon dioxide to release the gas into the environment. The second dialysis module can include a plurality of types of sorbent materials configured to regenerate the dialysate. Due to the heavier nature of the second dialysis module, it can be tethered, stationary, and can receive a power source such as through a power cord to a wall outlet.

The combined system for hemodialysis including the mobile first dialysis module 100 connected to the second dialysis module 200 and the auxiliary module 180 can keep the P-BUTS low, at a non-toxic level, during a shortened stationary period On the other hand, other substances known to be toxic, such as phosphorus and urea can be removed from the blood in sufficient amounts in 6-10 hours, thus not requiring longer periods for a sufficient removal. Urea and phosphorus are not P-BUTS. These can be removed using the bulkier second dialysis module 200.

A patient can alternate use of the first dialysis module and a second dialysis module, for example for about 12 hours each during a 24 hour period, such that the patient remains mobile during the day while maintaining overall desired removal of toxins. This can be done, for example, over a combined cycle for removal of a variety of toxins between the first dialysis module and the second dialysis module.

FIGS. 3A-3B illustrate a detachable sorbent cartridge canister 300 for use with any hemodialysis system disclosed herein. The canister 300 can have a top 302 and a bottom 304. The canister 300 can include a base 310, retention arms 312, one or more clips 314, a guiding feature 316, and one or more valves 318. FIGS. 3C-3H are perspective views of the detachable sorbent cartridge canister 300 for use with a hemodialysis system. The perspective views illustrate some of the canister retention action using the canister base 310, retention arms 312, one or more clips 314, guiding feature 316, and one or more valves 318.

The canister 300 can include a sorbent cartridge 320. The sorbent cartridge 320 can be within the canister 300, and include material that acts as a filter, absorbent, adsorbent, or catalyst, for removal or conversion of toxins in dialysate and blood pumped through the system 100. For example, the sorbent can be carbon, charcoal, zirconium phosphate, hydrous zirconium oxide, zirconium alloys, organic compounds containing zirconium, inorganic compounds containing zirconium, minerals containing zirconium, urease, or combinations thereof. In some cases, the canister 300 can include one sorbent cartridge 320 fitted to the inside of the canister. In some cases, the canister 300 can include more than one sorbent cartridge 320, of the same or differing types, to provide additional filtering capacity.

The retention arms 312, clips 314, a guiding feature 316, and valves 318 can allow for attachment and retention of the canister 300 in the system 100. For example, the retention arms 312 can allow for proper placement and alignment of the canister 300 within the system 100. The clips 314 can be opened and secured over the canister 300 and a protrusion or mating portion in the system 100. The guiding feature 316 can allow for proper alignment of the canister 300 in the system 100.

The canister 300 and the system 100 can include these features and more for proper alignment and connection of the canister 300 to the system 100. These features can allow attachment and detachment by an end-user into the system 100, such that the canister 300 can be readily swapped out for a canister hosting a different type of sorbent cartridge 320. These attachment/detachment features can include protrusions, locking, attachment, and snap features, discussed in more detail below. During attachment, the canister 300 can be attached to the system 100 but pressing the retention arms 312 to open them, inserting the canister 300 into the receptacle of the system 100, and then releasing the retention arms 312 so that they close on the protrusion locking in the canister 300. During removal, the release procedure is the opposite.

When connected to the system, these connection features can facilitate initial pressurization and canister swapping against a pressurized pump. For example, when the canister 300 is attached into the system 100 and into the dialysate circuit 150, the canister 300 is now in line with a fluid communication with the pump 120. The pump 120 can provide pressure to the system 100. Proper attachment and closure of the canister 300 can allow appropriate pressurization within the dialysate circuit 150 for filtering of dialysate/blood in the system 100 via the sorbent cartridge 320. In an example, the pressure in the canister 300 can be about 7 psi to about 15 psi, depending on the sorbent material used.

One or more sorbent cartridge 320 can be used. For example, the sorbent cartridge 320 can include a filter material with 5 to 7 mm filter, such as of a carbon compacted with aluminum. In another example, the sorbent cartridge can be a filter material with a stainless steel mess having about 0.0045 to about 0.0060 inch openings, such as a 1001-150 filter. In another sorbent cartridge, the filter material can be carbon, such as a charcoal or activated carbon in the amount of about 100 to about 200 g. In another example, the filter material can be a zirconium phosphate in an amount of about 250 to about 300 g, such as mixed with immobilized urease in an about of about 200 to about 300 g. In another example, the filter material can be a zirconium phosphate, such as mixed with a zirconium oxide, for example in the amount of about 330 g zirconium phosphate and about 220 g zirconium oxide. The filter materials in the sorbent cartridge can, for example, be secured with multi-layer screens. Separation of sorbent cartridge types into different canisters can allow for more efficient removal of toxins, prevent flow around filters, and prevent filters from migrating in canisters. This can additionally extend filter lifetime. The filters can be replaced within the canisters as desired.

The canister 300 can additionally include one or more caps or end sections configured to close the canister 300 at either end. The caps can, in some cases, be clear or see-through to allow monitoring of the canister. In some cases, this can allow for monitoring of bubbles within the canister. In some cases, a custom bubble filter can be included in the canister 300, such as to allow inspection for flow and preventing of bubble formations.

FIG. 3C depicts the canister clips 314 and base 310. The canister clips 314 can be used to secure the canister 300 to the system 100.

FIG. 3D depicts the valve 318. The valve 318 can be, for example, a Luer activated valve, such that the valve for receiving dialysate in the system 100 is only activated when the canister 300 is securely attached to the system 100. The valves 318 can prevent unwanted fluid loss when switching canisters. FIG. 3D also depicts the retention arms 312, which can be tensioned against system 100 when the canister is attached, and squeezed to allow release of the canister when desired.

FIG. 3E depicts the guiding feature 316 that can help an end user properly align the canister 300 with the system 100. The guiding feature 316 can prevent fitting damage while swapping canisters in and out of the system 100.

FIG. 3F shows the canister 300 clicked into place in the system 100 along with two other canisters. The canister 300 can be secured into the system 100 by pushing the canister into the receiving portion of the system 100, such as to click the clips and retention arms into place within the system 100. FIG. 3G and FIG. 3H show two sides of the canister 300 when attached into the system 100.

The canister 300 can optionally have a hinge mechanism for attachment to the system 100, one or more stiffening ribs to preserve the shape of the canister, and one or more O-ring or other sealing features to prevent leakage of dialysate. The canister 300 can be made through mechanical assembly, or through a jointless process such as three-dimensional printing.

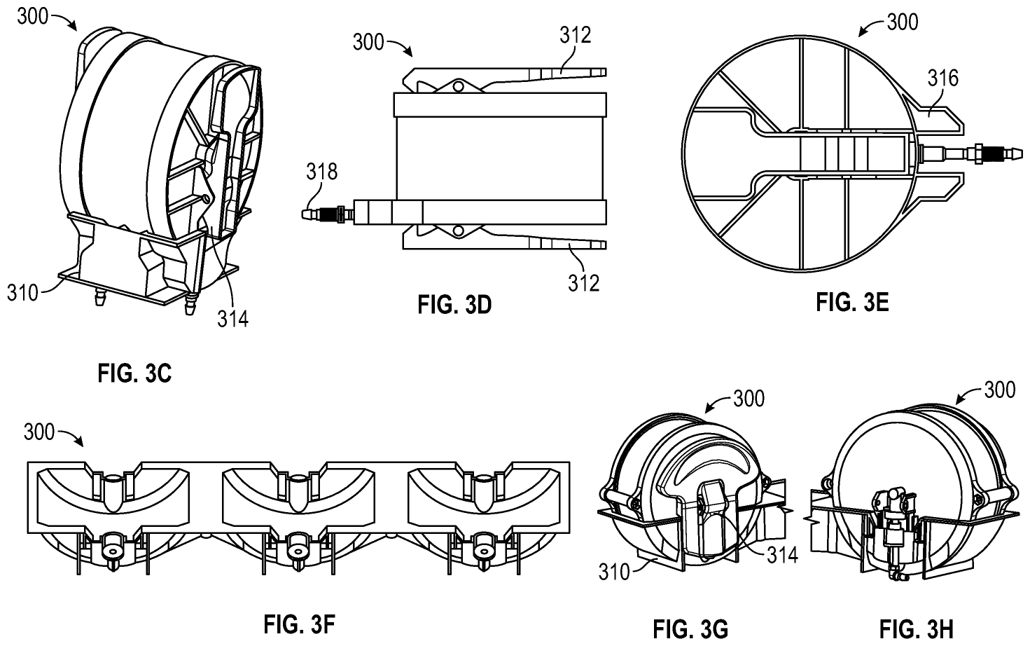

FIGS. 4A-4D are perspective exploded views of detachable sorbent cartridge canisters for use with a hemodialysis system and attachment fittings for detachably, mechanically securing a canister to the system 100. FIGS. 4A-4D show the canister 300 with various fittings 410, 420, 430, and 440. The fittings can allow for securing the canister 300 to the system 100 on the dialysate circuit, but can also allow end-user detachment and replacement of the canister 300.

In FIG. 4A, fitting 410 depicts a fitting where an end user would insert the canister 300 into the system 100 can twist to lock and connect the fitting 410 with a mating fitting in the system 100. The fitting 410 is a circular ring-like fitting with several flanges to engage a mating fitting.

In FIG. 4B, fitting 420 depicts a fitting where an end user would insert the canister 300 into the system 100 can twist to lock and engage the fitting 420 with a mating fitting in the system 100. The fitting 420 is a lateral fitting with two points of attachment for the canister into a mating fitting.

In FIG. 4C, fitting 430 depicts a fitting where an end user would insert the canister 300 into the system 100 can twist to lock the fitting 430 with a mating fitting in the system 100. The fitting 430 is a ring with two opposing locking portions.

In FIG. 4D, fitting 440 depicts a fitting where an end user would insert the canister 300 into the system 100 can clamp a partial ring around the canister 300 to lock and connect the fitting 440 with a mating fitting in the system 100. The fitting 440 is an open tension ring that clamps around the canister 300. For example, a user could spread open the legs of the fitting 440, slide the canister 300 into the rings, and then leg go of the legs to allow a snug fit of the canister 300 in the closed fitting 440.

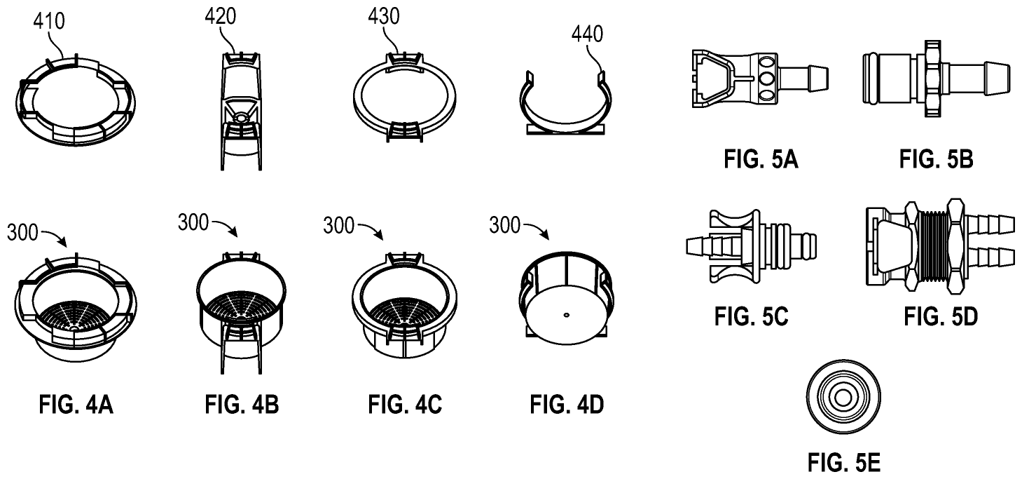

FIGS. 5A-5E are views of example connectors for detachable sorbent cartridge canisters that can be used to fluidly couple the canisters to a hemodialysis system. The connectors can, for example, include Luer fittings for fluidly and detachably coupling the canisters to the system 100, such as large bore barbed fittings, quick connect fittings, valved or non-valved fittings. The connectors in FIGS. 5A-5E can be for fluidly coupling the canisters to the system, so that dialysate in the dialysate circuit can flow through the canisters without leaking. In a given system 100, the canisters 300 can be detachably connected to and detached from the dialysate circuit through one or more of these types of connectors.

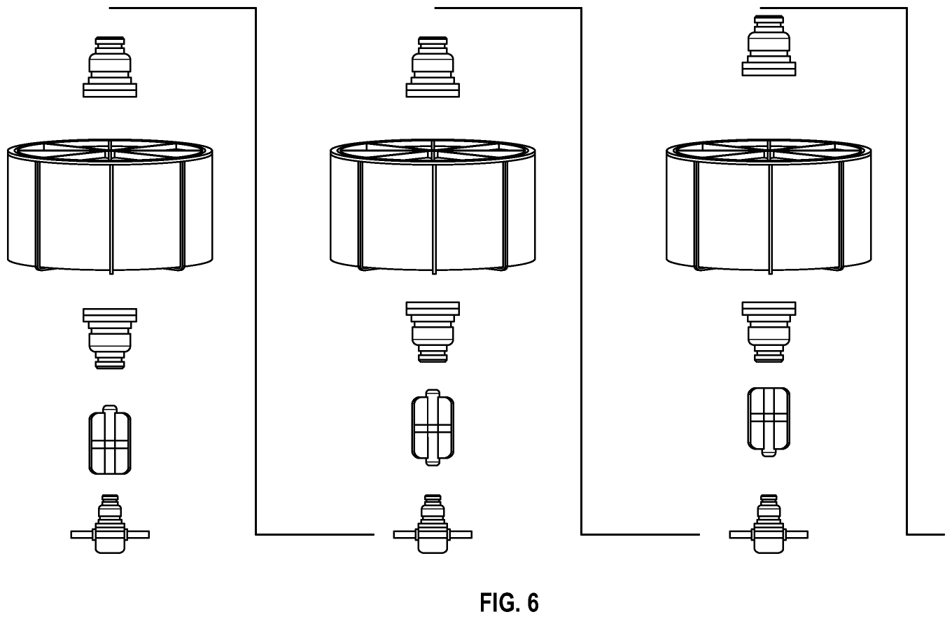

FIG. 6 is an exploded view of a series of detachable canisters 300. The cannisters 300 are shown connected in series with various connectors, such as those discuss with reference to FIGS. 5A-5E. Here, the dialysate in the system 100 can flow through all three canisters 300 in series. Each of the canisters 300 can have a different sorbent type, to allow filtering of various toxins as the dialysate runs through the canisters in series.

A side by side canister configuration allows for easier access and disconnection or replacement of individual canisters. For example, if the middle canister is not needed for a particular time period, it can be detached and removed, and the first and third canisters can be directly connected to each other. Conversely, if additional canisters, such as containing different or additional sorbent materials, are desired, they can be added in series. Each canister can be connected with one or more connectors and the appropriate tubing.

### Various Notes & Examples

Each of these non-limiting examples can stand on its own, or can be combined in various permutations or combinations with one or more of the other examples.

Example 1 can include a system for hemodialysis comprising a first dialysis module including a dialyzer, a blood circuit configured to receive blood from a patient, circulate the blood through the dialyzer, and return cleaned blood to the patient, a dialysate circuit configured to circulate dialysate through the dialyzer and remove impurities from the blood, and a first canister detachably coupled to dialysate circuit, the canister comprising a first sorbent.

Example 2 can include Examples 1, further comprising a second dialysis module detachably connectable to the first dialysis module, the second dialysis module comprising one or more hook-ups for connecting to the first dialysate module, and a second canister detachably coupled to dialysate circuit, the canister comprising a second sorbent for removing toxins from the blood.

Example 3 can include any of Examples 1-2, wherein the second dialysis module further comprises a third canister detachably coupled to the dialysate circuit.

Example 4 can include any of Examples 1-3, wherein the first sorbent comprises activated carbon for continuously removing the free fraction of protein bound uremic toxins.

Example 5 can include any of Examples 1-4, wherein the second sorbent comprises urease for removing urea from blood.

Example 6 can include any of Examples 1-5, wherein the first, second, and third canisters each comprise a sorbent chosen from the group comprising carbon, charcoal, zirconium phosphate, hydrous zirconium oxide, zirconium alloys, organic compounds containing zirconium, inorganic compounds containing zirconium, minerals containing zirconium, urease, or combinations thereof.

Example 7 can include any of Examples 1-6, wherein at least one of the first, second, and third canisters are detachably connected to the dialysate circuit by one or more mechanical connectors.

Example 8 can include any of Examples 1-7, wherein the one or more mechanical connectors include clips.

Example 9 can include any of Examples 1-8, the one or more mechanical connectors include a twisting interlock between the cannisters and the dialysate circuit.

Example 10 can include any of Examples 1-9, wherein at least one of the first, second, and third canisters includes one or more retention arms configured to secure the canisters in the dialysate circuit.

Example 11 can include a method of hemodialysis using a hemodialysis system. The method can include continuously removing a first set of toxins from blood over a combined cycle with a first dialysis module, and selectively removing a second set of toxins from the blood over a portion of the combined cycle with a second dialysis module, wherein removing the first set and the second set of toxins comprises using one or more detachable canisters fluidly connectable to the first dialysis module, the second dialysis module, or both.

Example 12 can include a method of hemodialysis using a hemodialysis system. The method can include releasably connecting at least one canister to the hemodialysis system, wherein the canister comprises at least one sorbent material for removal of toxins, selectively removing the toxins with the hemodialysis system including the at least one canister, and detaching the at least one canister from the hemodialysis system.

Example 13 can include Example 12, further comprising replacing the at least one canister with a second canister having a second sorbent material different than the first.

Example 14 can include any of Examples 12-13, further comprising releasably connecting a second canister to the hemodialysis system in series with the first at least one canister.

Example 15 can include any of Examples 12-14, wherein the second canister comprises a sorbent different than the first at least one sorbent.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for hemodialysis comprising:
a first dialysis module comprising:
a dialyzer;
a blood circuit configured to receive blood from a patient, circulate the blood through the dialyzer, and return cleaned blood to the patient;
a dialysate circuit configured to circulate dialysate through the dialyzer and remove impurities from the blood; and
a first canister detachably coupled to dialysate circuit, the canister comprising a first sorbent.

2. The system of claim 1, further comprising a second dialysis module detachably connectable to the first dialysis module, the second dialysis module comprising:
one or more hook-ups for connecting to the first dialysate module; and
a second canister detachably coupled to dialysate circuit, the canister comprising a second sorbent for removing toxins from the blood.

3. The system of claim 2, wherein the second dialysis module further comprises a third canister detachably coupled to the dialysate circuit.

4. The system of claim 1, wherein the first sorbent comprises activated carbon for continuously removing the free fraction of protein bound uremic toxins.

5. The system of claim 2, wherein the second sorbent comprises urease for removing urea from blood.

6. The system of claim 3, wherein the first, second, and third canisters each comprise a sorbent chosen from the group comprising carbon, charcoal, zirconium phosphate, hydrous zirconium oxide, zirconium alloys, organic compounds containing zirconium, inorganic compounds containing zirconium, minerals containing zirconium, urease, or combinations thereof.

7. The system of claim 3, wherein at least one of the first, second, and third canisters are detachably connected to the dialysate circuit by one or more mechanical connectors.

8. The system of claim 7, wherein the one or more mechanical connectors include clips.

9. The system of claim 7, the one or more mechanical connectors include a twisting interlock between the cannisters and the dialysate circuit.

10. The system of claim 7, wherein at least one of the first, second, and third canisters includes one or more retention arms configured to secure the canisters in the dialysate circuit.

11. A method of hemodialysis using a hemodialysis system, the method comprising:
continuously removing a first set of toxins from blood over a combined cycle with a first dialysis module;
selectively removing a second set of toxins from the blood over a portion of the combined cycle with a second dialysis module;
wherein removing the first set and the second set of toxins comprises using one or more detachable canisters fluidly connectable to the first dialysis module, the second dialysis module, or both.

12. A method of hemodialysis using a hemodialysis system, the method comprising:
releasably connecting at least one canister to the hemodialysis system, wherein the canister comprises at least one sorbent material for removal of toxins;
selectively removing the toxins with the hemodialysis system including the at least one canister; and
detaching the at least one canister from the hemodialysis system.

13. The method of claim 12, further comprising replacing the at least one canister with a second canister having a second sorbent material different than the first.

14. The method of claim 12, further comprising releasably connecting a second canister to the hemodialysis system in series with the first at least one canister.

15. The method of claim 14, wherein the second canister comprises a sorbent different than the first at least one sorbent.
